(19) United States
(12) Patent Application Publication
(10) Pub. No.: US 2026/0259122 A1
(43) Pub. Date: Sep. 3, 2026

(54) DISCHARGE DEVICE AND CONTROL METHOD THEREOF

(71) Applicant: LG INNOTEK CO., LTD., Seoul (KR)

(72) Inventor: Ji Na KWON, Seoul (KR)

(73) Assignee: LG INNOTEK CO., LTD., Seoul (KR)

(21) Appl. No.: 18/865,884

(22) PCT Filed: Jun. 9, 2023
(86) PCT No.: PCT/KR2023/007950

§ 371 Date: Nov. 14, 2024

(30) Foreign Application Priority Data
Jun. 9, 2022 (KR) 10-2022-0070345
Jul. 28, 2022 (KR) 10-2022-0093740

(51) Int. Cl. G01N 11/04; G01N 11/00; G01N 33/44

(52) U.S. Cl. CPC G01N 11/04; G01N 33/442; G01N 2011/008

(57) ABSTRACT

A discharge device according to an embodiment comprises a discharger for accommodating and discharging a polymer material; a sensor for sensing characteristics of the polymer material; and a controller for controlling a discharge pressure of the discharger according to a signal sensed from the sensor.

### TECHNICAL FIELD

The present invention relates to a discharge device, and more particularly, to a discharge device capable of changing discharge conditions by predicting a change on standing of a polymer material, and a method for controlling the same.

### BACKGROUND ART

In general, an adhesive member is used to bond a plurality of components together.

For example, a semiconductor package includes a circuit board including a semiconductor device and a cover disposed on the circuit board. The cover is also referred to a lid.

The cover can protect the semiconductor device placed on the circuit board and discharge heat generated from the circuit board and the semiconductor device to an outside.

At this time, the adhesive member is accommodated in a discharge device. In addition, the adhesive member is applied on the circuit board according to a discharge pressure controlled by the discharge device. The discharge pressure is determined based on an amount of the adhesive member applied or discharged. For example, the discharge device sets the discharge pressure based on the amount of the adhesive member to be applied or discharged on the circuit board.

Meanwhile, the adhesive member is composed of a polymer material such as epoxy.

In addition, viscosity or degree of cure of the polymer material changes depending on a surrounding environment. In addition, the change in the viscosity or degree of cure also occurs within the discharge device.

For example, the adhesive member accommodated in the discharge device may change in viscosity or degree of cure depending on the surrounding environment (e.g., temperature or humidity). In addition, the adhesive member accommodated in the discharge device may change in viscosity or degree of cure over time.

At this time, if the viscosity or degree of cure of the adhesive member changes, there is a problem that a certain amount of the adhesive member is not discharged from the discharge device.

For example, if the viscosity or degree of cure of the adhesive member increases while discharging a first amount of adhesive member with the discharge pressure of a first intensity, a second amount of adhesive member smaller than the first amount may be discharged from the discharge device. In addition, if the discharge amount of the adhesive member decreases as the viscosity or degree of cure increases, there is a problem that a bonding force between the circuit board and the cover decreases.

Meanwhile, the viscosity of the adhesive member can be measured using equipment that measures the viscosity of the adhesive member. For example, characteristics of the adhesive member can be analyzed using equipment that measures the viscosity of the adhesive member, and the viscosity of the adhesive member can be measured using this.

However, at least one hour or more is required for the characteristic analysis and viscosity measurement of the adhesive member. Therefore, there is a problem that it is difficult to measure the viscosity of the adhesive member in real time. That is, the viscosity of the adhesive member continues to change even while analyzing the adhesive member. Accordingly, the viscosity measured using an equipment means the viscosity of the adhesive member at a previous time, not the viscosity of the adhesive member at a current time. Accordingly, there is a problem in that it is difficult to check the viscosity or degree of cure of the polymer material in real time.

That is, conventionally, the viscosity of the adhesive member is measured for simple reference, and there is a problem in that it is difficult to measure the viscosity of the adhesive member in real time.

Therefore, there is a need for a method that can measure the viscosity of the adhesive member accommodated in the discharge device or discharged from the discharge device in real time is required.

### DISCLOSURE

### Technical Problem

The embodiment provides a discharge device capable of measuring the viscosity or degree of cure of an adhesive member in real time and a method for controlling the same.

In addition, the embodiment provides a discharge device capable of measuring the viscosity or degree of cure of an adhesive member according to a state of a functional group included in the adhesive member and a method for controlling the same.

In addition, the embodiment provides a discharge device capable of precisely analyzing characteristics of an adhesive member in real time and a method for controlling the same.

In addition, the embodiment provides a discharge device capable of improving characteristics of a process of discharging an adhesive member and a method for controlling the same.

In addition, the embodiment provides a discharge device capable of quantitatively predicting a change on standing of an adhesive member and a method for controlling the same.

In addition, the embodiment provides a discharge device capable of discharging a constant amount of an adhesive member to a workpiece regardless of time or temperature change and a method for controlling the same.

Technical problems to be solved by the proposed embodiments are not limited to the above-mentioned technical problems, and other technical problems not mentioned can be clearly understood by those skilled in the art to which the embodiments proposed from the following descriptions belong.

### Technical Solution

A discharge device according to an embodiment comprises a discharger for accommodating and discharging a polymer material; a sensor for sensing characteristics of the polymer material; and a controller for controlling a discharge pressure of the discharger according to a signal sensed from the sensor.

In addition, the polymer material includes an adhesive member.

In addition, the characteristics of the adhesive member sensed by the sensor are information corresponding to viscosity.

In addition, the information corresponding to the viscosity is information on a functional group.

In addition, the information on the functional group is information on an amine group.

In addition, the adhesive member is a room temperature curing adhesive member.

In addition, the adhesive member is a two-component adhesive member.

In addition, the sensor senses the characteristics of the adhesive member discharged from the discharger.

In addition, the sensor senses the characteristics of the adhesive member accommodated in the discharger.

In addition, the sensor acquires information of the adhesive member that reacts to an infrared light source.

In addition, the sensor includes an output unit outputting the infrared light source, and a receiving unit receiving reflected light of the infrared light source reflected from the adhesive member.

In addition, the sensor receives transmission light of the infrared light source that passes the adhesive member.

In addition, the discharger includes a first region for accommodating a first material, a second region for accommodating a second material different from the first material, and a third region in which the first material and the second material are mixed.

In addition, the sensor senses the adhesive member of the third region.

Meanwhile, a method for controlling discharge according to an embodiment includes discharging a polymer material; sensing a characteristic of the polymer material; and controlling a discharge pressure based on the sensed information.

In addition, the discharged polymer material includes an adhesive member.

In addition, the sensed information is information corresponding to a viscosity or a degree of cure of the adhesive member.

In addition, the controlling of the discharge pressure includes increasing the discharge pressure as the viscosity or degree of cure is measured higher.

In addition, the sensed information is information about a functional group of the adhesive member, and the controlling of the discharge pressure includes controlling the discharge pressure according to a sensing amount of the functional group of the adhesive member.

In addition, the controlling of the discharge pressure includes increasing the discharge pressure when the sensing amount decreases.

In addition, the information about the functional group is information about an amine group included in the adhesive member.

### Advantageous Effects

The embodiment can measure the viscosity or degree of cure of the adhesive member in real time. For example, the embodiment can measure the viscosity or degree of cure of the adhesive member according to a state of the functional group included in the adhesive member. Accordingly, the embodiment can precisely analyze the characteristics of the adhesive member in real time. In addition, the embodiment can quantitatively predict the change on standing of the adhesive member.

In addition, the embodiment can control a discharge condition of the discharger according to the change in the viscosity or degree of cure of the adhesive member. For example, when the viscosity or degree of cure of the adhesive member increases, the embodiment can increase the discharge pressure of the discharger corresponding thereto.

Therefore, the embodiment can improve the discharge process characteristics of the adhesive member.

Furthermore, the embodiment can discharge a constant amount of the adhesive member to a workpiece regardless of time or temperature changes.

Accordingly, the embodiment can improve product reliability.

### DESCRIPTION OF DRAWINGS

FIG. 1 is a schematic diagram of a discharge system according to an embodiment.

FIG. 2 is a diagram for explaining a detailed structure of a discharger of FIG. 1.

FIG. 3 is a diagram for explaining a detailed structure of a sensor of FIG. 1.

FIG. 4 is a diagram showing an example of an infrared spectrum acquired by the sensor of FIG. 3.

FIG. 5 is a diagram for explaining a reaction between a first material and a second material of an adhesive member according to an embodiment.

FIG. 6 is a diagram for explaining a change in viscosity of an adhesive member over time according to an embodiment.

FIG. 7 is a diagram showing a change in state of a functional group of an adhesive member over time.

FIG. 8 is a diagram showing a relationship between a number of functional groups and the viscosity of an adhesive member according to an embodiment.

FIG. 9 is a flowchart showing a control method of a discharge device according to an embodiment step by step.

### BEST MODE

Hereinafter, embodiments of the present disclosure will be described in detail with reference to the accompanying drawings.

However, the spirit and scope of the present disclosure is not limited to a part of the embodiments described, and can be implemented in various other forms, and within the spirit and scope of the present disclosure, one or more of the elements of the embodiments can be selectively combined and redisposed.

In addition, unless expressly otherwise defined and described, the terms used in the embodiments of the present disclosure (including technical and scientific terms) can be construed the same meaning as commonly understood by one of ordinary skill in the art to which the present disclosure belongs, and the terms such as those defined in commonly used dictionaries can be interpreted as having a meaning that is consistent with their meaning in the context of the relevant art. In addition, the terms used in the embodiments of the present disclosure are for describing the embodiments and are not intended to limit the present disclosure.

In this specification, the singular forms can also include the plural forms unless specifically stated in the phrase, and can include at least one of all combinations that can be combined in A, B, and C when described in “at least one (or more) of A (and), B, and C”. Further, in describing the elements of the embodiments of the present disclosure, the terms such as first, second, A, B, (a), and (b) can be used.

These terms are only used to distinguish the elements from other elements, and the terms are not limited to the essence, order, or order of the elements. In addition, when an element is described as being “connected”, “coupled”, or “contacted” to another element, it can include not only when the element is directly “connected” to, “coupled” to, or “contacted” to other elements, but also when the element is “connected”, “coupled”, or “contacted” by another element between the element and other elements.

In addition, when described as being formed or disposed “on (over)” or “under (below)” of each element, the “on (over)” or “under (below)” can include not only when two elements are directly connected to each other, but also when one or more other elements are formed or disposed between two elements. Further, when expressed as “on (over)” or “under (below)”, it can include not only the upper direction but also the lower direction based on one element.

Hereinafter, a discharge device and a control method thereof according to an embodiment will be described.

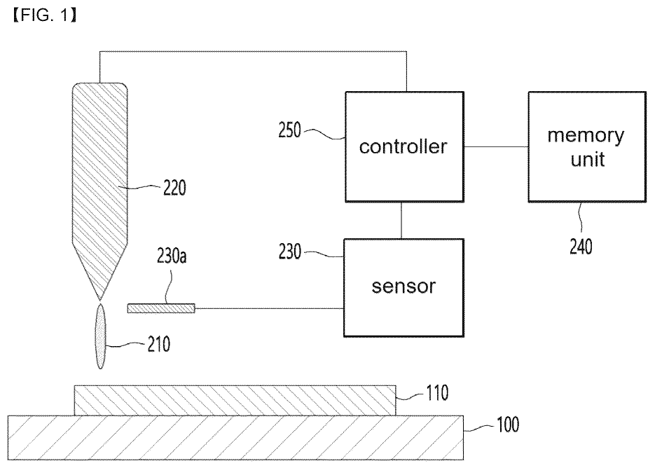

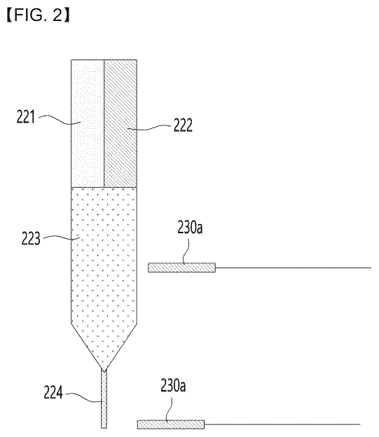

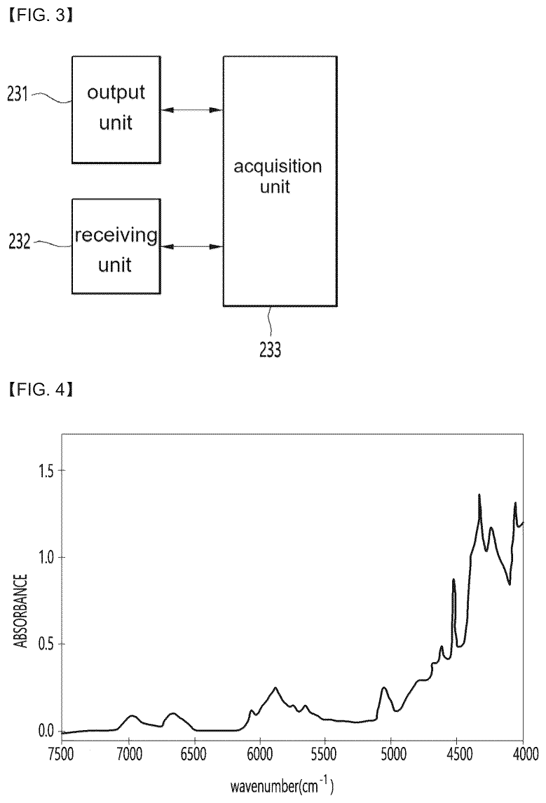

FIG. 1 is a schematic diagram of a discharge system according to an embodiment, FIG. 2 is a diagram for explaining a detailed structure of a discharger of FIG. 1, FIG. 3 is a diagram for explaining a detailed structure of a sensor of FIG. 1, and FIG. 4 is a diagram showing an example of an infrared spectrum acquired by the sensor of FIG. 3.

Referring to FIG. 1, a discharge system includes a work table 100, a workpiece 110, and a discharge device 200.

The work table 100 can provide a space where the workpiece 110 is placed. For example, the work table 100 can provide the workpiece 110 to a region where the discharge device 200 is placed.

The work table 100 can include a moving part that moves the workpiece 110 to a discharge region and a fixing part that fixes the workpiece 110 moved to the discharge region.

The workpiece 110 can be placed on the work table 100. The workpiece 110 can mean a circuit board to which an adhesive member is to be applied by the discharge device 200. However, the embodiment is not limited thereto. For example, the workpiece 110 may be a component of a camera module to which an adhesive member other than a circuit board is to be applied.

The discharge device 200 may apply an adhesive member on the workpiece 110 placed on the work table 100.

To this end, the discharge device 200 may include a discharger 220 that accommodates the adhesive member 210 and discharges the accommodated adhesive member 210 to the workpiece 110.

The adhesive member 210 may be a polymer material. For example, the adhesive member 210 may be a polymer material including an adhesive component. As an example, the adhesive member 210 may be an epoxy resin, but is not limited thereto.

In other words, the adhesive member 210 is a polymer material including an adhesive component, and may include an epoxy resin. The epoxy resin is one of a thermosetting resins, and can have excellent properties of withstanding water and weather changes and excellent curing and adhesive properties.

The epoxy resin constituting the adhesive member 210 may be selected from a group including bisphenol A type epoxy resin, bisphenol F type epoxy resin, bisphenol S type epoxy resin, alicyclic epoxy resin, cresol novolac type epoxy resin, and biphenyl type epoxy resin. Hereinafter, the adhesive member 210 will be described as being an epoxy resin, which is one of the polymer materials having an adhesive material.

In one embodiment, the adhesive member 210 may be a one-component adhesive member. For example, a main agent and a curing agent may be accommodated in a mixed state in the discharger 220. In the one-component adhesive member, the main agent and the hardening agent may hardly react at room temperature. For example, the one-component adhesive member may be a heat-curing type adhesive member or a UV-curing type adhesive member.

In another embodiment, the adhesive member 210 may be a two-component adhesive member. For example, the main agent and the curing agent may be accommodated in a separate state in the discharger 220. Then, the discharger 220 may mix the main agent and the curing agent and discharge them to the workpiece 110. For example, the two-component adhesive member may be a room temperature curing type adhesive member. That is, since the room temperature curing type adhesive member has a very high reactivity between the main agent and the curing agent, a rate of change in viscosity or degree of cure is high over time. Therefore, the main agent and the curing agent may be accommodated in a separate state in the discharger 220. Then, when it is desired to discharge the adhesive member 210 to the workpiece 110, the main agent and the curing agent may be mixed and a mixed material may be discharged to the workpiece 110.

Meanwhile, the discharge device 200 of the embodiment can sense a change in characteristics of the adhesive member 210 accommodated in the discharger 220 or the adhesive member 210 being discharged from the discharger 220 and control discharge conditions of the discharger 220 according to the sensed change in the characteristics of the adhesive member 210.

At this time, as described above, in the case of a one-component adhesive member, the reactivity is very low at room temperature. Therefore, if the adhesive member 210 accommodated in the discharger 220 is a one-component adhesive member, there may be almost no change in the characteristics of the adhesive member 210. Accordingly, if the adhesive member 210 is a one-component adhesive member, the discharge condition control of the discharge device 200 according to the embodiment may be unnecessary. For example, if the adhesive member 210 is a one-component adhesive member, an effect exhibited by the discharge device 200 of the embodiment may be minimal. For example, in a case of a one-component adhesive member, since the reactivity at room temperature is very low, there may be little change in the degree of cure or viscosity.

However, although the one-component adhesive member does not react well at room temperature, it is stored frozen in preparation for an emergency. For example, the one-component adhesive member is stored at a temperature of about −20 degrees. In addition, in order to use the one-component adhesive member, a process is performed in which the frozen one-component adhesive member is exposed to room temperature for a certain period of time. However, the viscosity or degree of cure may change depending on the surrounding environment during the process in which the frozen one-component adhesive member melts. In addition, although the one-component adhesive member has low reactivity at room temperature, a change on standing may occur depending on time or the surrounding environment.

Therefore, the embodiment can control the discharge conditions according to the change in the characteristics of the adhesive member 210 described below for the one-component adhesive member. Accordingly, the embodiment senses the change on standing of the single-component adhesive member, thereby preventing an adhesive member 210 of less than a target amount from being discharged onto the workpiece 110.

However, the discharge device 200 of the embodiment can be maximized when applied to a two-component adhesive member. For example, since the two-component adhesive member is highly reactive at room temperature, the change in the characteristics of the main agent and the curing agent mixed in the discharger 220 is very large depending on time or the surrounding environment. Therefore, the embodiment senses the change in the characteristics of the adhesive member 210 accommodated in the discharger 220 or the adhesive member 210 discharged from the discharger 220, and controls the discharge condition of the discharger 220 based on this.

Accordingly, a following description will be made for a case where the adhesive member 210 is a two-component adhesive member. However, the embodiment is not limited thereto, and the adhesive member 210 accommodated in the discharger 220 and discharged by the discharger 220 may be a one-component adhesive member rather than a two-component adhesive member.

The adhesive member 210 may include a first material corresponding to the main agent and a second material corresponding to the curing agent. The first material may be referred to as an epoxy resin corresponding to the main agent. In addition, the second material may be referred to as the curing agent.

The first material may mean an epoxy resin corresponding to the main agent. The first material may enable the adhesive member 210 to have a certain level or higher of adhesive strength through curing and adhesive actions.

A type of the first material is not particularly limited as long as it includes at least two or more functional groups. Specifically, the first material may include a functional group that meets and reacts with the second material. For example, a functional group included in the first material may include an epoxy group or an amine group derived from an epoxy group. The functional group may also be expressed as a cross linker, a binder, a terminal group, a reactive group, etc.

Specifically, the first material may be any one of a bisphenol A type epoxy resin, a bisphenol F type epoxy resin, a bisphenol S type epoxy resin, a phenol novolac type epoxy resin, a cresol novolac type epoxy resin, a bisphenol A novolac type epoxy resin, a bisphenol F novolac type epoxy resin, an alicyclic epoxy resin, a glycidyl ester type epoxy resin, a glycidyl amine epoxy resin, a hydantoin type epoxy resin, an isocyanate type epoxy resin, an aliphatic chain epoxy resin, and a terminal amine-modified epoxy resin.

The second material may be a material for polymerizing the first material. For example, the first material may be a low-molecular weight material before meeting the second material. In addition, the first material may be polymerized by meeting and reacting with the second material. Therefore, a mixed material in which the first material and the second material are mixed may be a polymerized polymer material when the first material and the second material meet and react. Specifically, as the first material and the second material are mixed, the amine group and the epoxide group corresponding to the functional group meet and react. In addition, when the amine group and the epoxide group meet and react with each other, a molecular weight of the polymer material increases, and the characteristics of the polymer material can change as much as the increase in molecular weight.

A type of the second material is not particularly limited, and may include, for example, an amine-based curing agent, a phenol-based curing agent, an acid anhydride (also called an anhydride, an oxide formed by dehydrating an acid from an inorganic acid or separating one molecule of water through a condensation reaction of a carboxylic acid group from an organic acid)-based curing agent, a hydrazide-based curing agent, dicyandiamide, etc.

In addition, examples of amine-based curing agents include polyoxyalkylene polyamines, polyamides, amidoamines, aliphatic amines, tertiary amines, aromatic aliphatic amines, cycloaliphatic amines, aromatic amines, isophorone diamine, etc. In addition, phenol-based curing agents include phenol novolac, cresol novolac, bisphenol A novolac, and halogenated compounds of novolac resins. These may be used alone or in combination of two or more. In addition, examples of the acid anhydride curing agent may include at least one selected from the group consisting of methylhexahydrophthalic anhydride, methyl tetrahydrophthalic anhydride, methyl-5-norbornene-2,3-dicarboxylic anhydride, hexahydrophthalic anhydride, tetrahydrophthalic anhydride, dodecylsuccinic anhydride, phthalic anhydride, and succinic anhydride.

Preferably, the first material of the embodiment may be DGEBA (Diglycidyl ether of Bisphenol A, a bisphenol A type liquid epoxy resin) represented by the chemical formula 1 below, and the second material may be 1-(2-Aminoethyl) piperazine represented by the chemical formula 2 below.

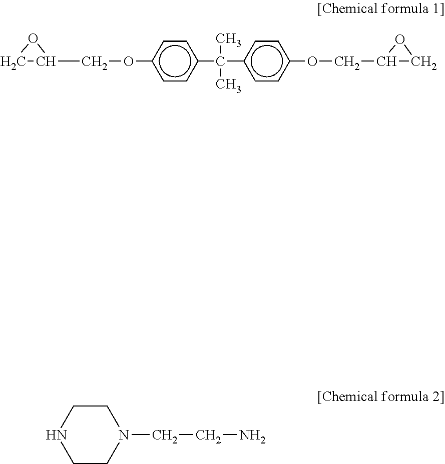

Meanwhile, the adhesive member 210 may further include a filler. The filler may be added to the first material and the second material to improve the mechanical properties of the adhesive member 210. The filler may have functions such as reducing costs, reducing thermal expansion rate, reducing curing shrinkage rate, controlling heat generation during curing, improving adhesion, imparting thixotropic properties, imparting flame retardancy, imparting chemical resistance, improving thermal conductivity, increasing mechanical strength, improving electrical properties, and improving wear resistance.

The adhesive member 210 may be accommodated in the discharger 220. In addition, the adhesive member 210 may be discharged from the discharger 220 to the workpiece 110.

To this end, the discharger 220 may provide an accommodation space in which the adhesive member 210 is accommodated. At this time, if the adhesive member 210 is a one-component adhesive member, the accommodation space of the discharger 220 may not be divided into multiple regions. However, when the adhesive member 210 is a two-component adhesive member, the accommodation space of the discharge device 200 may be divided into multiple regions.

Referring to FIG. 2, the accommodation space of the discharger 220 may be divided into multiple regions.

For example, the discharger 220 may include a first region 221, a second region 222, and a third region 223. Specifically, the discharge device 200 may include a syringe divided into multiple regions while accommodating the adhesive member 210.

The first region 221 of the syringe may provide a space in which the first material of the adhesive member 210 is accommodated. The first region 221 may be divided with the second region 222.

The second region 222 of the syringe can provide a space in which the second material of the adhesive member 210 is accommodated. The second region 222 can be divided from the first region 221.

That is, the first material is accommodated in the first region 221, and the second material is accommodated in the second region 222. In addition, in a state in which the first material is accommodated in the first region 221 and the second material is accommodated in the second region 222, the first material and the second material do not meet each other.

In addition, the discharger 220 further includes a third region 223 of the syringe. The third region 223 can be connected to the first region 221 and the second region 222, respectively. For example, the third region 223 may be a region where the first material accommodated in the first region 221 and the second material accommodated in the second region 222 meet. For example, the third region 223 may be a region where the first material of the first region 221 and the second material of the second region 222 are mixed with each other.

That is, in a case of the two-component adhesive member, it may be separately accommodated in the first region 221 and the second region 222 of the discharger 220. In addition, when discharge of the adhesive member 210 is required to the workpiece 110, the first material and the second material may move to the third region 223 and be mixed with each other. In addition, the mixed material may be discharged through a needle 224 of the discharger 220.

For example, the discharger 220 of the embodiment may be a needle-type dispenser for discharging the adhesive member 210, but is not limited thereto.

As described above, the two-component adhesive member may be separated into the first region 221 and the second region 222 of the discharger 220, mixed in the third region 223, and then discharged through the needle 224.

Meanwhile, the discharger 220 may include an air inlet passage provided at an upper portion of the syringe, although not shown in the drawing. In addition, compressed air may be introduced through the air inlet passage. The intensity of the compressed air may be controlled by a controller to be described later. In addition, the discharge amount of the adhesive member 210 discharged through the needle 224 of the discharger 220 may be controlled by the intensity of the compressed air controlled by the controller.

At this time, the two-component adhesive member is a room temperature curing adhesive member. That is, the two-component adhesive member can be cured by the reaction of the first material of the main agent and the second material of the curing agent at room temperature.

Here, the “room temperature” may specifically mean a temperature in a state of not being heated or cooled. For example, the room temperature may mean a temperature within a range of 10° C. to 30° C. Specifically, the room temperature may mean a temperature of 15° C. or higher, 18° C. or higher, 20° C. or higher, or 23° C. or higher, and 27° C. or lower.

In addition, since the two-component adhesive member is a room temperature curing type, the first material and the second material can meet and react with each other at room temperature. In addition, the characteristics of the adhesive member 210, which is a mixture of the first material and the second material, may change depending on the degree of reaction between the first material and the second material. Here, the characteristics may mean the viscosity of the adhesive member 210. For example, the characteristic may mean the degree of cure of the adhesive member 210.

At this time, the first material and the second material may meet and react in the third region 223 of the discharger 220. In addition, the viscosity or degree of cure, which is a characteristic of the adhesive member 210 mixed in the third region 223, may change over time.

At this time, if the adhesive member 210 mixed in the third region 223 is used within a short period of time or in a continuous process, there may not be a significant change in the discharge amount of the adhesive member 210 according to the viscosity or degree of cure.

However, the adhesive member 210 present in the discharger 220 is generally used for 24 hours or more. In addition, during the process of discharging the adhesive member 210, the discharge process may not proceed for a certain period of time due to reasons such as inspection of other equipment. Accordingly, the viscosity or degree of cure of the adhesive member 210 existing in the third region 223 may change over time.

Furthermore, not only the adhesive member 210 accommodated in the third region 223, but also the viscosity or degree of cure of the adhesive member 210 discharged through the needle 224 may change. For example, the needle 224 includes metal. Then, the adhesive member 210 discharged from a tip of the needle 224 including the metal may meet with air, thereby increasing the reactivity of the adhesive member 210, and thus the viscosity or degree of cure may change.

At this time, if the viscosity or degree of cure of the adhesive member 210 changes, the discharge amount of the adhesive member 210 discharged from the discharger 220 under same discharge conditions may change.

For example, the discharge amount of the adhesive member 210 discharged in a state where the viscosity or degree of cure is high is smaller than the discharge amount of the adhesive member 210 discharged in a state where the viscosity or degree of cure is low.

Therefore, a constant discharge amount of the adhesive member 210 should always be discharged from the discharger 220, but the viscosity or degree of cure of the adhesive member 210 changes depending on the degree of reaction between the first material and the second material of the adhesive member 210, and thus the discharge amount of the adhesive member 210 discharged from the discharger 220 changes.

Accordingly, the embodiment senses a change in the characteristics of the adhesive member 210 accommodated in the third region 223 of the discharger 220 and/or the adhesive member 210 discharged from the needle 224 of the discharger 220.

To this end, the discharge device 200 of the embodiment may include a sensor 230.

The sensor 230 may sense the characteristics of the adhesive member 210. Specifically, the sensor 230 may sense the viscosity of the adhesive member 210. For example, the sensor 230 may sense the degree of cure of the adhesive member 210.

To this end, the sensor 230 may sense a state of the functional group provided in the adhesive member 210. For example, the sensor 230 can sense a state of the functional group provided in the adhesive member 210.

That is, the characteristic of the adhesive member 210 can be a state of the functional group of the adhesive member 210, and the state of the functional group of the adhesive member 210 can mean the viscosity or degree of cure of the adhesive member 210.

In addition, the embodiment senses a state of the functional group of the adhesive member 210 and senses the characteristic such as the viscosity or degree of cure of the adhesive member 210 based on the state of the functional group.

To this end, the sensor 230 can sense the characteristic of the adhesive member 210 using a FT-IR (Fourier Transform Infrared) analysis method.

Here, the FT-IR (Fourier Transform Infrared) analysis method is a method of analyzing the characteristic of a sample by using infrared (IR) irradiated to the sample. When infrared is irradiated to the sample, molecules in the sample absorb infrared of a specific frequency and vibrate due to the infrared. In addition, this can be expressed as a characteristic infrared spectrum corresponding to an energy by the vibration of molecules in the sample. Accordingly, by analyzing the infrared spectrum, information on the molecules in the sample can be acquired, and various information contained in the spectrum can be utilized.

In addition, the sensor 230 can include a probe 230a for sensing. The probe 230a can be a sensing mechanism for sensing the characteristics of the adhesive member 210 without changing the characteristics of the adhesive member 210 as a measurement target. The probe 230a can be a non-contact sensing mechanism.

The probe 230a of the sensor 230 can sense the characteristics of the adhesive member 210 accommodated in the third region 223 of the syringe of the discharger 220. In another embodiment, the probe 230a can sense the characteristics of the adhesive member 210 being discharged through the needle 224 of the discharger 220. In another embodiment, the probe 230a is provided in multiple numbers, and thus can sense the characteristics of the adhesive member 210 accommodated in the third region 223 and the characteristics of the adhesive member 210 being discharged through the needle 224.

Specifically, the viscosity or degree of cure of the adhesive member 210 can change depending on the degree of reaction between the first material and the second material. For example, the viscosity or degree of cure can also increase as the degree of mutual reaction between the first material and the second material increases. In addition, the degree of mutual reaction between the first material and the second material can be confirmed based on a state of the functional group provided in the first material and the second material.

For example, when the degree of reaction between the first material and the second material is low (for example, when no reaction occurs), a functional group of a first intensity exists in the adhesive member 210. In addition, when the first material and the second material react with each other, the functional group provided in the adhesive member 210 decreases to a second intensity lower than the first intensity.

Accordingly, the embodiment senses an intensity of the functional group of the adhesive member 210 and senses the viscosity or degree of cure of the adhesive member 210 based on the intensity of the functional group. A relationship between the intensity of the functional group and the viscosity/degree of cure of the adhesive member 210 is described in more detail below.

To this end, referring to FIG. 3, the sensor 230 may include an output unit 231, a receiving unit 232, and an acquisition unit 233.

The output unit 231 may include an infrared light source. That is, the output unit 231 may be referred to as an infrared light source generating unit that generates an infrared light source. The output unit 231 may irradiate an infrared light source to a sensing target.

In one embodiment, the output unit 231 may irradiate an infrared light source to the third region 223 of the syringe of the discharger 220.

In another embodiment, the output unit 231 may irradiate an infrared light source to an adhesive member 210 being discharged through the needle 224 of the discharger 220.

At this time, the output unit 231 may irradiate an infrared light source of a specific band. The infrared light source may be a near infrared ray, or alternatively, a mid-infrared ray.

Preferably, the infrared light source may be a near infrared ray.

That is, the adhesive member 210 includes a filler. In addition, when the output unit 231 uses mid-infrared light, energy by the filler may be reflected in the spectrum acquired from the sensor 230. Accordingly, noise may be included in a peak value corresponding to the functional group in the infrared spectrum acquired from the sensor 230.

In contrast, when the output unit 231 uses near-infrared light, the energy by the filler may not be reflected in the infrared spectrum acquired from the sensor 230, and thus the accuracy of the peak value corresponding to the functional group may be high. Specifically, when the output unit 231 uses near-infrared light, quantitative analysis of the functional group provided in the adhesive member 210 may be possible using the spectrum sensed by the sensor 230.

The receiving unit 232 may receive infrared light reacted by the adhesive member 210.

In one embodiment, the receiving unit 232 may receive reflected light reflected by the adhesive member 210.

In another embodiment, the receiving unit 232 may receive transmission light transmitted through the adhesive member 210.

The acquisition unit 233 can acquire a characteristic infrared spectrum by the vibration of molecules in the adhesive member 210 using the reflected light or transmission light received through the receiving unit 232.

For example, referring to FIG. 4, the infrared spectrum can be expressed as absorption according to wavenumber. In addition, the embodiment can sense a state of the functional group by using absorption of a specific frequency to be analyzed in the infrared spectrum. This will be described in more detail below.

The discharge device of the embodiment includes a memory unit 240. The memory unit 240 can store information necessary for the operation of the discharge device. The memory unit 240 can store information generated during the operation of the discharge device.

Preferably, the memory unit 240 can store a program for processing or controlling the controller 250 and various information for an overall operation of the discharge device by the controller 250. For example, the memory unit 240 can store information on the relationship between the infrared spectrum sensed by the sensor 230 and the characteristics of the adhesive member 210. For example, the memory unit 240 can store information on the relationship between the intensity of a specific frequency in the infrared spectrum and the viscosity or degree of cure of the adhesive member 210 corresponding thereto.

The memory unit 240 can be various storage devices such as ROM, RAM, EPROM, flash drive, hard drive, etc. in terms of hardware.

The controller 250 can control the overall operation of the discharge device.

The controller 250 controls the discharge condition of the discharger 220 so that a certain amount of the adhesive member 210 is discharged to the workpiece 110.

In addition, the controller 250 periodically senses information on the characteristics of the adhesive member 210 through the sensor 230. For example, the controller 250 acquires an infrared spectrum for the adhesive member 210 through the sensor 230.

In addition, the controller 250 can control the discharge condition of the discharger 220 according to the characteristics of the adhesive member 210 sensed based on the infrared spectrum sensed by the sensor 230. Here, the discharge condition may include a discharge pressure. However, the embodiment is not limited thereto. For example, the discharge condition may include a discharge time. Hereinafter, the discharge condition will be described as being a discharge pressure.

For example, the controller 250 can measure the viscosity or degree of cure of the adhesive member 210 using the infrared spectrum.

In addition, the controller 250 can adjust the discharge pressure of the discharger 220 when the measured viscosity or degree of cure changes. For example, the controller 250 can increase the discharge pressure of the discharger 220 when a high viscosity or a high degree of cure is measured.

In other words, the controller 250 can detect information on the functional group included in the adhesive member 210 using the infrared spectrum. For example, the controller 250 can detect an area of intensity or a height of intensity in a certain wavelength band corresponding to the functional group included in the adhesive member 210 based on the infrared spectrum. In addition, the detected information can mean a number of functional groups included in the adhesive member 210. Thereafter, the controller 250 increases the discharge pressure of the discharger 220 as the detected amount of the functional group included in the adhesive member 210 decreases.

The embodiment can measure the viscosity or degree of cure of the adhesive member in real time. For example, the embodiment can measure the viscosity or degree of cure of the adhesive member according to a state of the functional group included in the adhesive member. Accordingly, the embodiment can precisely analyze the characteristics of the adhesive member in real time. In addition, the embodiment can quantitatively predict the change on standing of the adhesive member.

In addition, the embodiment can control a discharge condition of the discharger according to the change in the viscosity or degree of cure of the adhesive member. For example, when the viscosity or degree of cure of the adhesive member increases, the embodiment can increase the discharge pressure of the discharger corresponding thereto.

Therefore, the embodiment can improve the discharge process characteristics of the adhesive member.

Furthermore, the embodiment can discharge a constant amount of the adhesive member to a workpiece regardless of time or temperature changes.

Accordingly, the embodiment can improve product reliability.

Hereinafter, the change in the viscosity or degree of cure of the adhesive member over time and the relationship between the viscosity or degree of cure and the functional group included in the adhesive member 210 are described.

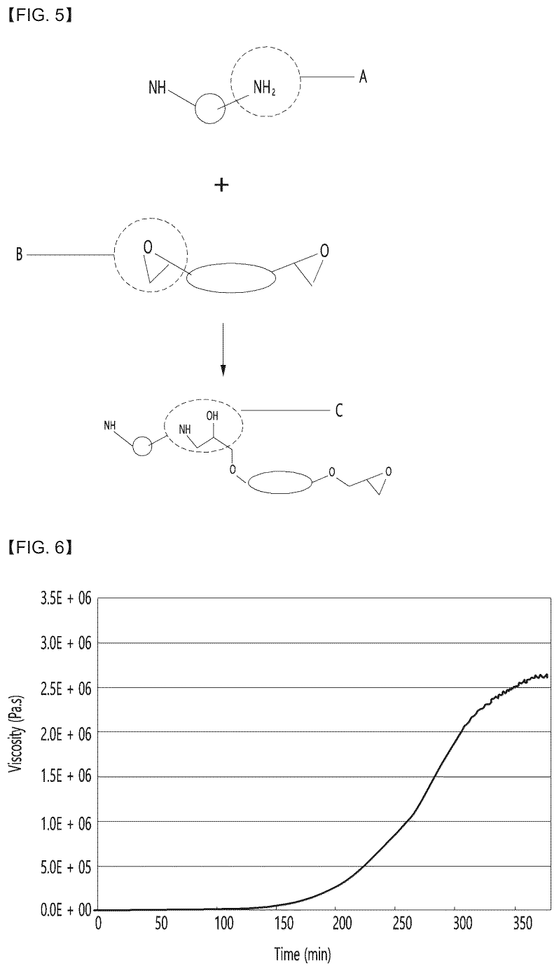

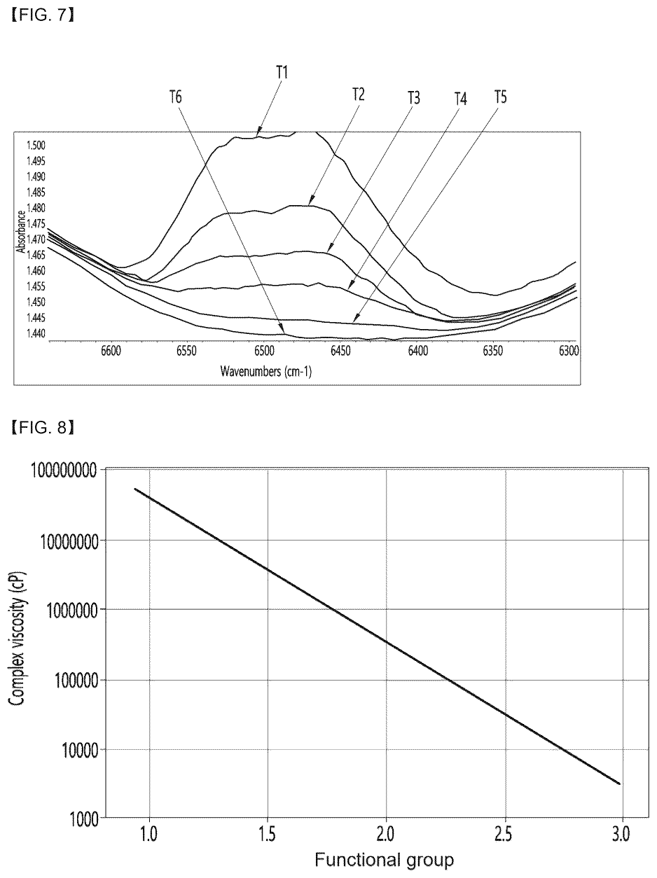

FIG. 5 is a drawing for explaining the reaction of the first material and the second material of the adhesive member according to an embodiment, FIG. 6 is a drawing for explaining the viscosity change of the adhesive member over time according to an embodiment, FIG. 7 is a drawing showing a state change of the functional group of the adhesive member over time, and FIG. 8 is a drawing showing the relationship between the number of functional groups and the viscosity of the adhesive member according to an embodiment.

Referring to FIG. 5, the adhesive member 210 of the embodiment may include the first material and the second material as described above. In addition, the first material and the second material may each include a functional group.

The functional group may vary depending on a type of material constituting the first material and the second material.

For example, the first material may be provided with an epoxide group (O, A of FIG. 5), and the first material may be provided with an amine group (NH₂, B of FIG. 5).

In addition, when the first material and the second material meet, the epoxide group (O, A in FIG. 5) and the amine group (NH₂, B in FIG. 5)) can react with each other. In addition, when the epoxide group (O, A of FIG. 5) and the amine group ((NH₂, B of FIG. 5) react with each other, the amine group (NH₂, B of FIG. 5) changes to a state such as NH (C of FIG. 5).

In other words, the adhesive member 210 includes an amine group (NH₂). In addition, the amine group (NH₂) changes to NH depending on the degree of reaction of the adhesive member 210.

Accordingly, a fact that the amine group (NH₂) changes to NH may mean that the degree of mutual reaction between the first material and the second material in the adhesive member 210 is high. Furthermore, a fact that the degree of reaction is high means that the viscosity or degree of cure of the adhesive member 210 increases.

In summary, a fact that the amine group (NH₂), which is a functional group provided in the adhesive member 210, changes to NH means that the viscosity or degree of cure of the adhesive member 210 has increased. Furthermore, a decrease in a number of amine groups (NH₂) provided in the adhesive member 210 may also mean that the viscosity or degree of cure of the adhesive member 210 has increased.

Meanwhile, the functional group provided in the adhesive member 210 is amine group (NH₂) as an example, but it is not limited thereto. For example, the functional group of the adhesive member 210 may be a thiol group (SH) instead of the amine group (NH₂) depending on the types of the first material and the second material.

Meanwhile, referring to FIG. 6, the viscosity (viscosity) of the adhesive member 210 may change over time. For example, the viscosity of the adhesive member 210 may also increase as time increases.

Meanwhile, referring to FIG. 7, the number of functional groups provided in the adhesive member 210 may decrease over time. That is, the viscosity of the adhesive member 210 increases over time, and further, a number of functional groups decreases. The number of functional groups may be expressed as absorbance in the infrared spectrum.

That is, in the embodiment, it was confirmed how the number of functional groups provided in the adhesive member 210 changes over time. At this time, the embodiment shows an infrared spectrum for an adhesive member 210 including an amine group (NH₂) over time.

At this time, the amine group (NH₂) can react at a wavelength of 6625 (cm-1) in the infrared spectrum.

Therefore, the information of the amine group (NH₂) included in the adhesive member 210 can be confirmed by analyzing the information of the wavelength of 6625 (cm-1) in the infrared spectrum.

In addition, as shown in FIG. 7, it was confirmed that the number of amine groups (NH₂) included in the adhesive member 210 gradually decreases over time.

For example, it can be seen that the absorbance of the wavelength of 6625 (cm-1) in the infrared spectrum sensed at a first time (T1) is approximately 1.500.

For example, it can be seen that the absorbance of the wavelength band of 6625 (cm-1) in the infrared spectrum sensed at a second time (T2) after the first time (T1) is approximately 1.475.

For example, it can be seen that the absorbance of the wavelength band of 6625 (cm-1) in the infrared spectrum sensed at a third time (T3) after the second time (T2) is approximately 1.465.

For example, it can be seen that the absorbance of the wavelength band of 6625 (cm-1) in the infrared spectrum sensed at a fourth time (T4) after the third time (T3) is approximately 1.455.

For example, it can be seen that the absorbance of the wavelength band of 6625 (cm-1) in the infrared spectrum sensed at a fifth time (T5) after the fourth time (T4) is approximately 1.445.

For example, it can be seen that the absorbance of the wavelength band of 6625 (cm-1) in the infrared spectrum sensed at a sixth time (T6) after the fifth time (T5) is approximately 1.440.

That is, as time passes (for example, from T1 to T6), the degree of reaction of the first material and the second material included in the adhesive member 210 may increase. In addition, as the degree of reaction increases, the number of functional groups provided in the adhesive member 210 may decrease. Therefore, when the number of functional groups decreases, the absorbance of the wavelength band corresponding to the functional group in the sensed infrared spectrum may decrease. In addition, the embodiment can measure the viscosity or degree of cure of the adhesive member 210 based on the absorbance of the wavelength band corresponding to the functional group in the infrared spectrum.

Meanwhile, as shown in FIG. 8, the embodiment confirmed the relationship between the number of amine groups (NH₂) included in the adhesive member 210 and the viscosity of the adhesive member 210 corresponding thereto.

As shown in FIG. 8, when the number of functional groups is small, it was confirmed that the viscosity of the adhesive member 210 corresponding thereto is high. Furthermore, when the number of functional groups is large, it was confirmed that the viscosity corresponding thereto is low.

Therefore, the embodiment stores the relationship information between the detection amount of the functional group in the adhesive member 210 and the viscosity of the adhesive member 210 corresponding thereto in the memory unit 240. In addition, the controller 250 controls to acquire an infrared spectrum from the sensor 230 according to a certain period. Here, the period may be 3 seconds, but is not limited thereto.

In addition, the controller 250 can analyze information of a wavelength band corresponding to a functional group of the adhesive member 210 in the acquired infrared spectrum, and measure the viscosity or degree of cure of the adhesive member 210 based on the information. Accordingly, the controller 250 can adjust the discharge pressure of the discharger 220 based on the viscosity or degree of cure.

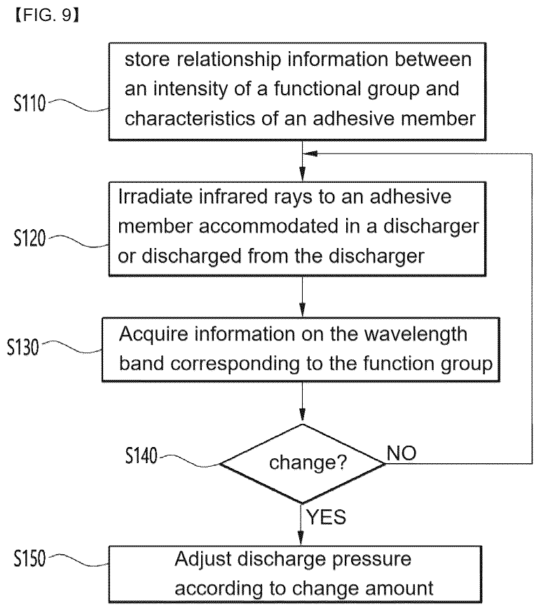

FIG. 9 is a flowchart showing a control method of a discharge device according to an embodiment step by step.

Referring to FIG. 9, the embodiment stores relationship information of an intensity of a functional group included in the adhesive member 210 (for example, area or detection amount or height of a peak value) and the characteristic of the adhesive member 210 corresponding to the intensity of the functional group (S110). The characteristic of the adhesive member 210 may represent the viscosity or degree of cure of the adhesive member 210.

Next, the sensor 230 irradiates infrared rays to the adhesive member 210 accommodated in the discharger 220 or discharged from the discharger 220 (S120).

Thereafter, the sensor 230 acquires an infrared spectrum according to the infrared irradiation. Then, the sensor 230 can transmit the acquired infrared spectrum to the controller 250.

Next, the controller 250 can analyze the infrared spectrum to measure the characteristics of the adhesive member 210 (S130). For example, the controller 250 can analyze an area of a specific wavelength band or a height of a peak value in the infrared spectrum. Then, the controller 250 can measure the detection amount of the functional group provided in the adhesive member 210 based on the area of the specific wavelength band or the height of the peak value. In addition, the controller 250 can measure the viscosity or degree of cure of the adhesive member 210 based on the detection amount of the functional group.

Next, the controller 250 can determine whether the measured viscosity or degree of cure has changed (S140).

In addition, if the viscosity or degree of cure has changed, the controller 250 can adjust the discharge condition of the discharger 220 corresponding to the amount of change in the viscosity or degree of cure (S150). For example, the controller 250 can increase the discharge pressure of the discharger 220 in proportion to the amount of change in the viscosity or degree of cure of the adhesive member 210.

The embodiment can measure the viscosity or degree of cure of the adhesive member in real time. For example, the embodiment can measure the viscosity or degree of cure of the adhesive member according to a state of the functional group included in the adhesive member. Accordingly, the embodiment can precisely analyze the characteristics of the adhesive member in real time. In addition, the embodiment can quantitatively predict the change on standing of the adhesive member.

In addition, the embodiment can control a discharge condition of the discharger according to the change in the viscosity or degree of cure of the adhesive member. For example, when the viscosity or degree of cure of the adhesive member increases, the embodiment can increase the discharge pressure of the discharger corresponding thereto.

Therefore, the embodiment can improve the discharge process characteristics of the adhesive member.

Furthermore, the embodiment can discharge a constant amount of the adhesive member to a workpiece regardless of time or temperature changes.

Accordingly, the embodiment can improve product reliability.

Features, structures, effects, etc. described in the above embodiments are included in at least one embodiment, and it is not necessarily limited to only one embodiment. Furthermore, features, structures, effects, etc. illustrated in each embodiment can be combined or modified for other embodiments by those of ordinary skill in the art to which the embodiments belong. Accordingly, the contents related to such combinations and variations should be interpreted as being included in the scope of the embodiments.

In the above, the embodiment has been mainly described, but this is only an example and does not limit the embodiment, and those of ordinary skill in the art to which the embodiment pertains will appreciate that various modifications and applications not illustrated above are possible without departing from the essential characteristics of the present embodiment. For example, each component specifically shown in the embodiment can be implemented by modification. In addition, the differences related to these modifications and applications should be interpreted as being included in the scope of the embodiments set forth in the appended claims.

1-10. (canceled)

11. A discharge device comprising:
a discharger for receiving and discharging a polymer material;
a sensor for sensing characteristics of the polymer material; and
a controller for controlling a discharge pressure of the discharger according to a signal sensed from the sensor, and
wherein the sensor senses a number of a functional group of the polymer material and senses the characteristics of the polymer material.

12. The discharge device of claim 11, wherein the polymer material includes an adhesive member.

13. The discharge device of claim 12, wherein the characteristics of the adhesive member sensed by the sensor are information corresponding to viscosity.

14. The discharge device of claim 11, wherein the discharger includes:
a first region for accommodating a first material,
a second region for accommodating a second material different from the first material, and
a third region in which the first material and the second material are mixed.

15. The discharge device of claim 13, wherein the information on the functional group is information on an amine group.

16. The discharge device of claim 12, wherein the adhesive member is a room temperature curing adhesive member.

17. The discharge device of claim 12, wherein the adhesive member is a two-component adhesive member.

18. The discharge device of claim 12, wherein the sensor senses the characteristics of the adhesive member discharged from the discharger.

19. The discharge device of claim 12, wherein the sensor senses the characteristics of the adhesive member accommodated in the discharger.

20. The discharge device of claim 12, wherein the sensor acquires information of the adhesive member that reacts to an infrared light source.

21. The discharge device of claim 20, wherein the sensor includes an output unit outputting the infrared light source, and a receiving unit receiving reflected light from the infrared light source reflected from the adhesive member.

22. The discharge device of claim 20, wherein the sensor receives transmission light from the infrared light source that passes the adhesive member.

23. The discharge device of claim 14, wherein the sensor senses the adhesive member accommodated in the third region.

24. A method of controlling discharge comprising:
discharging a polymer material;
sensing characteristics of the polymer material; and
controlling a discharge pressure based information about the sensed characteristics,
wherein the sensing of the characteristics includes sensing a number of a functional group included in the discharged polymer material.

25. The method of claim 24, wherein the discharged polymer material includes an adhesive member.

26. The method of claim 25, wherein the information is information corresponding to a viscosity or curing degree of the adhesive member.

27. The method of claim 26, wherein the controlling of the discharge pressure includes increasing the discharge pressure as the viscosity or curing degree is measured to be higher.

28. The method of claim 26, wherein the controlling of the discharge pressure includes controlling the discharge pressure according to a detection amount of the number of the functional group of the adhesive member.

29. The method of claim 28, wherein the controlling of the discharge pressure includes increasing the discharge pressure when the detection amount decreases.

30. The method of claim 28, wherein the detection amount of the number of the functional group is information about an amine group included in the adhesive member.
